(19) United States
(12) Patent Application Publication
(10) Pub. No.: US 2023/0102317 A1
(43) Pub. Date: Mar. 30, 2023

(54) POLYMER MEDICAMENT FOR TREATING HYPERKALEMIA AND PREPARATION METHOD THEREOF

(71) Applicant: WATERSTONE PHARMACEUTICALS (WUHAN) CO., LTD., Wuhan (CN)

(72) Inventors: Min FU, Wuhan (CN); Huiling TANG, Wuhan (CN); Tongtong LI, Wuhan (CN); Minglong HU, Wuhan (CN); Kang BIE, Wuhan (CN); Ying LIANG, Wuhan (CN); Jian CUI, Wuhan (CN); Faming ZHANG, Wuhan (CN)

(21) Appl. No.: 17/696,225

(22) Filed: Mar. 16, 2022

Related U.S. Application Data
(63) Continuation of application No. PCT/CN2021/118053, filed on Sep. 13, 2021.

(51) Int. Cl. C08G 8/14; A61K 31/795; A61K 31/765; A61K 45/06; A61P 3/02

(52) U.S. Cl. CPC C08G 8/14; A61K 31/795; A61K 31/765; A61K 45/06; A61P 3/02

(57) ABSTRACT

Provided are a polymer medicament for treating hyperkalemia, and a preparation method thereof. Specifically, a polymer is provided, and the polymer includes repeating units obtained by polymerizing a monomer and a crosslinking agent. A molar ratio of the monomer to the crosslinking reagent ranges from 1:0.02 to 1:0.20. The monomer includes an acidic group and a pKa-reducing group next to the acidic group. The acidic group is selected from the group consisting of sulfonic acid group (—SO₃—), sulfuric acid group (—OSO₃—), carboxylic group (—CO₂—), phosphonic acid group (—OPO₃²—), phosphate group (—OPO₃²—), and sulfamic acid group (—NHSO₃—). The pKa-reducing group is selected from the group consisting of nitro, cyano, carbonyl, trifluoromethyl, and halogen atoms. The crosslinking agent has three or four reaction sites. The polymer can be used to treat hyperkalemia.

### CROSS-REFERENCE TO RELATED APPLICATIONS

The present application is a continuation of International Application No. PCT/CN2021/118053 filed on Sep. 13, 2021, the entire content of which is incorporated herein by reference.

### FIELD

The present disclosure relates to the field of medicinal chemistry, and particularly, the present disclosure relates to a polymer medicament for treating hyperkalemia and a preparation method thereof.

### BACKGROUND

Potassium (K⁺) is the most abundant cation in cells, and the content thereof in the human body is about 35 mEq/kg to 40 mEq/kg. Serum potassium in a range of about 5.0 mEq/L to 6.0 mEq/L can be defined as mild hyperkalemia, which is usually not life threatening. However, the moderate to severe hyperkalemia (serum potassium greater than about 6.1 mEq/L) may cause serious consequences. Arrhythmia and ECG waveform distortion are both the characteristics of hyperkalemia. When the serum potassium level rises to about 9 mEq/L or higher, symptoms such as atrioventricular dissociation, ventricular tachycardia, or ventricular fibrillation may occur.

Hyperkalemia is rare in the general healthy population. However, for some populations, hyperkalemia has a higher incidence. Among hospitalized patients, the incidence of hyperkalemia is about 1% to 10%, depending on the definition of hyperkalemia. Severe patients, premature babies or the elderly all belong to the groups at higher risk. Decreased kidney function, genitourinary diseases, cancer, severe diabetes, and mixed administration may all increase the risk of hyperkalemia in patients.

Most of the existing therapeutic regimens for hyperkalemia are limited to hospitalization. Ion-exchange resins, such as Kayexalate, are not suitable for outpatients or long-term treatment due to the fact that large doses must be used and the patients are reluctant to cooperate. Such a treatment has serious side effects on the gastrointestinal (GI) tract and may introduce excess sodium, which potentially leads to hypernatremia, related fluid retention and hypertension. Diuretics can allow patients to eliminate sodium and potassium through the kidneys. Nevertheless, due to the existing nephropathy and related diuretic resistance, the efficacy of diuretics is often limited. In addition, the diuretics are contraindicated in those patients who do not want to have a drop in blood pressure and volume. For example, CHF patients have hypotension, and are often administered with a combination of drugs, using ACE inhibitors and non-potassium diuretics that may induce hyperkalemia, such as spironolactone.

Therefore, it is urgent to develop a new drug with higher binding capacity for treating hyperkalemia.

### SUMMARY

In an aspect of the present disclosure, the present disclosure provides a polymer. According to an embodiment of the present disclosure, the polymer includes repeating units obtained by polymerizing a monomer and a crosslinking agent. A molar ratio of the monomer to the crosslinking agent ranges from 1:0.02 to 1:0.20. The monomer includes an acidic group and a pKa-reducing group next to the acidic group. The acidic group is selected from the group consisting of sulfonic acid group (—SO₃—), sulfuric acid group (—OSO₃—), carboxylic group (—CO₂—), phosphonic acid group (—OPO₃²—), phosphate group (—OPO₃²—), and sulfamic acid group (—NHSO₃—). The pKa-reducing group is selected from the group consisting of nitro, cyano, carbonyl, trifluoromethyl, and halogen atoms. The crosslinking agent has three or four reaction sites. Applicant found that the polymer according to the embodiment of the present disclosure has extremely high stability and potassium ion adsorption capacity when being in an acid state rather than a salt state. The polymer according to the embodiment of the present disclosure can be used as a medicament for the effective treatment of hyperkalemia.

According to an embodiment of the present disclosure, the above-mentioned polymer may further include at least one of the following technical features.

According to an embodiment of the present disclosure, the acidic group is the carboxylic group, and the pKa-reducing group is fluorine.

According to an embodiment of the present disclosure, the reaction sites are free alkenyl groups.

According to an embodiment of the present disclosure, the crosslinking agent includes at least one of triallyl isocyanurate, 1,2,4-trivinylcyclohexane, tetravinylsilane, trimethylolpropane trimethacrylate, triallyl cyanurate, or triallylcarbinol.

According to an embodiment of the present disclosure, the polymer is at least one selected from the group consisting of polyvinyl sulfonic acid polymer, polyvinyl sulfamic acid polymer, poly(vinyl sulfamic acid/vinyl sulfuric acid) copolymer, polyvinyl amino phosphonic acid polymer, N-(bisphosphonate ethyl) polyvinylamine polymer, poly(α-fluoroacrylic acid) polymer, vinylphosphonic acid/acrylic acid copolymer, vinylphosphonic acid/α-fluoroacrylic acid copolymer, polyvinylsulfuric acid polymer, and cross-linked polyvinylsulfamic acid polymer.

In another aspect of the present disclosure, the present disclosure further provides a polymer. According to an embodiment of the present disclosure, the polymer has a structure represented by formula (I) or is a salt of the structure represented by formula (I):

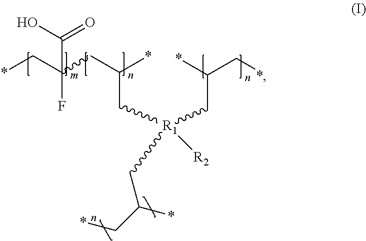

in which, R₁ is

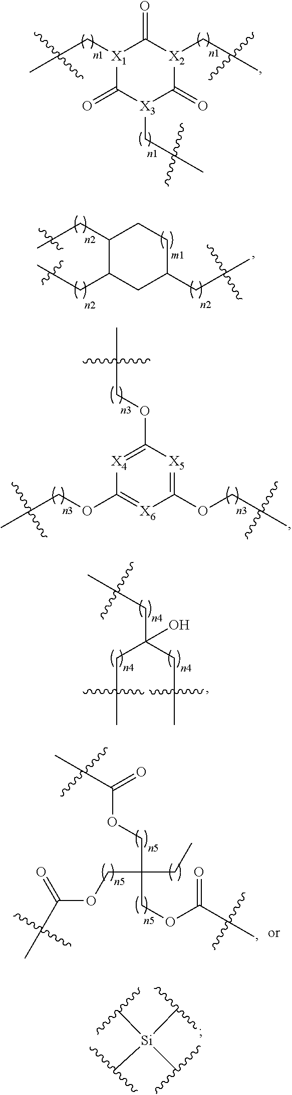

when R₁ contains three binding sites, R₂ is not present; and when R₁ contains four binding sites, R₂ is connected to one of the four binding sites of R₁ and R₂ is

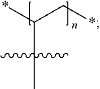

m ranges from 0.80 to 0.98, n ranges from 0.02 to 0.20, and m+n=1;
n₁, n₂, n₃, n₄, and n₅ are each independently selected from 0, 1, 2, 3, 4, 5, 6, or 7;
X₁, X₂, X₃, X₄, X₅, and X₆ are each independently a carbon atom or a nitrogen atom;
m1 is 0 or 1; and
represents a binding site.

According to an embodiment of the present disclosure, the above-mentioned polymer may further include at least one of the following technical features.

According to an embodiment of the present disclosure, R₁ is

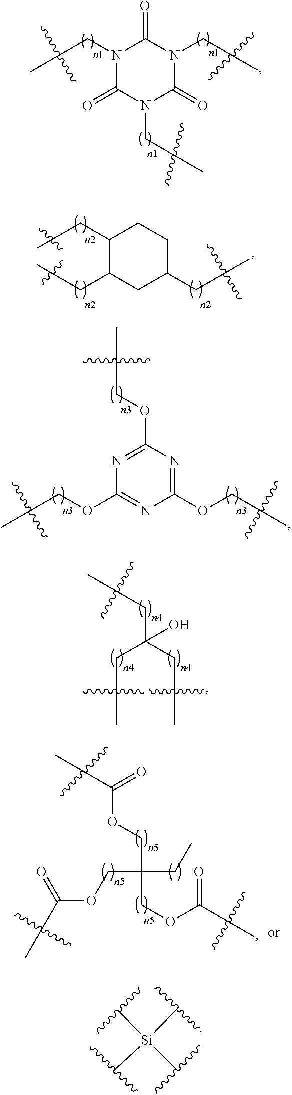

According to an embodiment of the present disclosure, R₁ is

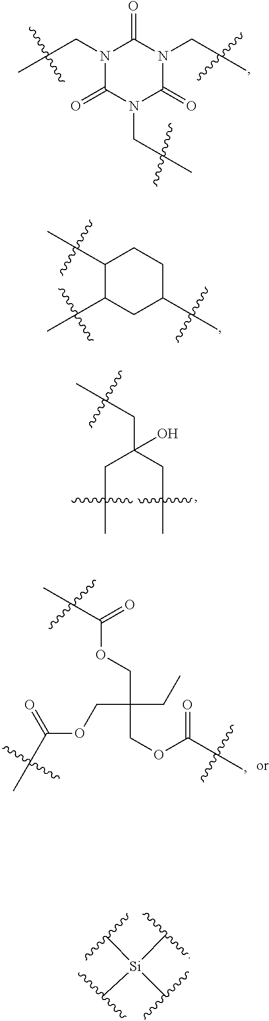

In yet another aspect of the present disclosure, the present disclosure further provides a polymer. According to an embodiment of the present disclosure, the polymer has any one of the following structures or is a salt of any one of the following structures:

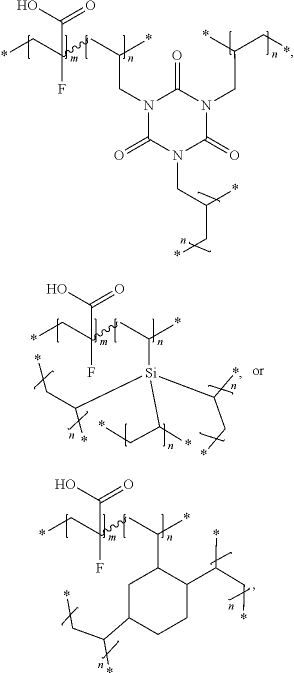

in which m ranges from 0.80 to 0.98; n ranges from 0.02 to 0.20; and m+n=1.

In yet another aspect of the present disclosure, the present disclosure further provides a pharmaceutical composition. According to an embodiment of the present disclosure, the pharmaceutical composition includes the polymer as described above, and a pharmaceutically acceptable excipient.

In yet another aspect of the present disclosure, the present disclosure further provides a use of the polymer as described above or the composition as described above in manufacture of a medicament for adsorbing potassium.

In yet another aspect of the present disclosure, the present disclosure further provides a use of the polymer as described above or the composition as described above in manufacture of a medicament for treating or preventing hyperkalemia.

According to an embodiment of the present disclosure, the above-mentioned use may further include at least one of the following technical features.

According to an embodiment of the present disclosure, the hyperkalemia is caused by administration of a medicament that causes potassium retention.

According to an embodiment of the present disclosure, the medicament that causes potassium retention is selected from the group consisting of non-potassium diuretics, angiotensin-converting enzyme inhibitors, non-steroidal anti-inflammatory drugs, heparin, and trimethoprim.

According to an embodiment of the present disclosure, the present disclosure further provides a method for preparing the above-mentioned polymer. According to an embodiment of the present disclosure, the method includes: adding a monomer, a crosslinking agent, an initiator, a dispersant, and an inorganic salt to water, dissolving and dispersing them uniformly at room temperature, reacting for a period of time at a certain temperature to obtain a solid of the polymer, then removing an alkyl moiety from the polymer to generate a polymer carboxylate, and finally acidifying the polymer carboxylate with an acidic solution to obtain the finished product.

A ratio of the monomer to the crosslinking agent ranges from 1:0.02 to 1:0.20, and when the ratio is 1:0.026, the sample has a higher adsorption capacity for potassium ions.

In some embodiments, both a water-soluble free radical initiator and an oil-soluble free radical initiator can be used to initiate the reaction to obtain the corresponding polymer. The water-soluble initiator includes, but is not limited to, potassium persulfate, ammonium persulfate, 2,2′-azobis(2-methylpropionamidine)dihydrochloride (V50), 2,2′-azabis(2-imidazoline) dihydrochloride (VA044), etc.

The above-mentioned oil-soluble initiator includes, but is not limited to, 2,2′-azobis(2-methylpropionitrile), 2,2′-azobis-(2,4-dimethylvaleronitrile), 2,2-azodi(2-methylbutyronitrile), 1,1′-azobis(cyclohexane-1-carbonitrile), dimethyl 2,2′-azobis(2-methylpropionate), dibenzoyl peroxide, lauroyl peroxide, cumene hydroperoxide, etc.

The dispersant used in certain embodiments includes, but is not limited to, gelatin, polyvinyl alcohol, sodium carboxymethyl cellulose, hydroxymethyl cellulose, sodium polyacrylate, calcium carbonate, magnesium carbonate, barium sulfate, diatomite, Talc powder, Tween 20, Tween 40, Tween 80, Tween 85, Span 20, Span 40, Span 60, Span 65, Span 80, Span 85, etc.

The inorganic salt used in certain embodiments includes, but is not limited to, potassium chloride, sodium chloride, ammonium chloride, calcium chloride, magnesium chloride, etc.

A method for removing alkyl group from the polymer includes hydrolysis using an alkali, and the used alkali includes, but is not limited to, potassium hydroxide, sodium hydroxide, lithium hydroxide, magnesium hydroxide, potassium carbonate, sodium carbonate, etc.

The hydrolysis of the present disclosure is carried out in a mixed solution of an aqueous alkali solution and an organic solvent. The used organic solvent includes, but is not limited to, ethanol, methanol, isopropanol, toluene, acetonitrile, diethyl ether, tetrahydrofuran, etc. It is surprisingly found that during removing of the alkyl moiety using ether solvents, the polymer product prepared with tetrahydrofuran and other ethers has better properties and colors. The ether used includes, but is not limited to, tetrahydrofuran, methyl tert-butyl ether, dimethoxyethane, ethylene glycol diethyl ether, etc.

The acid used in the above acidification of the polymer includes, but is not limited to, sulfuric acid, hydrochloric acid, nitric acid, etc.

In yet another aspect of the present disclosure, the present disclosure further provides a potassium ion adsorption determination method. According to an embodiment of the present disclosure, the method includes: detecting an adsorption capacity of the polymer as described above for potassium ions by ion chromatography.

According to an embodiment of the present disclosure, the conditions of the ion chromatography are as below:

Chromatographic column: IonPac CS17 Analytical Column (4×250 mm)

Protection column: IonPac CG17 Guard Column (4×50 mm)

Flow rate: 1.0 m1/min

Detector: Electrical conductivity detector

Column temperature: 30° C.

Injection volume: 10 μl

Eluent: 6 mM methanesulfonic acid solution
100421 Running time: 20 min.

Definition and Explanation

Those skilled in the art can understand that the *binding site can be further connected to monomers or the same or different crosslinking agents.

The present disclosure provides a method, a composition and a kit for treating animals. As used herein, “test animals” and “animals” include humans and other mammals. The present disclosure specifically provides a polymer composition for eliminating potassium ions. Preferably, the composition can be used to eliminate potassium ions from the gastrointestinal tract of animals.

A part of the present disclosure relates to a method for eliminating potassium using a potassium-binding polymer composition. In a certain embodiment, the potassium-binding polymer composition has high potassium-binding energy and/or selectivity, and can release the unbound potassium in the gastrointestinal tract. Preferably, the potassium-binding polymer composition will not release the bound potassium in the rectum. The polymer composition of the present disclosure has binding selectivity for potassium ions.

Preferably, the polymer composition of the present disclosure has a high binding energy and/or selectivity to potassium. The “high binding energy” herein means that each gram of the polymer can bind about 1.5 mmol or more of potassium in the body on average, and the in vivo potassium-binding energy herein is usually determined in vivo. Techniques for measuring the in vivo potassium-binding energy are known. For example, after a patient is given a potassium-binding polymer, the content of potassium in the patient's faeces can be used to calculate the in vivo potassium-binding energy. Optimally, an average potassium-binding energy in the human body is obtained through experiments and calculations based on a group of normal subjects. The group of subjects includes about 5 members, preferably about 10 members, more preferably about 25 members, and most preferably about 50 members.

The potassium-binding energy of the polymers of the present disclosure can also be determined in vitro. Preferably, the in vitro determination of the potassium-binding energy of the polymer of the present disclosure is carried out under the physiological conditions simulating gastrointestinal tract, especially the colon. In certain embodiments, the in vitro determination of the potassium-binding energy of the polymers of the present disclosure is performed in a solution having a pH of about 5.5 or higher. In various embodiments, the potassium-binding energy determined in a solution having a pH of about 5.5 or higher is equal to or greater than 6 mmol/g. Preferably, the in vitro potassium-binding energy of the polymer, which is determined in a solution having a pH of about 5.5 or higher, is between about 6 mmol/g and about 12 mmol/g. More preferably, the in vitro potassium-binding energy of the polymer, which is determined in a solution having a pH of about 5.5 or higher, is equal to or greater than about 6 mmol/g. More preferably, the in vitro potassium-binding energy of the polymer is equal to or greater than about 8 mmol/g. More preferably, the in vitro potassium-binding energy of the polymer is equal to or greater than about 10 mmol/g. Most preferably, the in vitro potassium-binding energy of the polymer is equal to or greater than about 12 mmol/g.

If the polymer composition has high potassium-binding energy, the dose of the composition can be reduced. Generally, the effective therapeutic and preventive dose of the polymer composition ranges from about 0.5 g/day and about 25 g/day. The most preferred dose is equal to or less than about 15 g/day. A preferred dose range is between about 5 g/day to about 20 g/day. A more preferred dose range is between about 5 g/day to about 15 g/day. A more preferred dose range is between about 10 g/day to about 20 g/day. The most preferred dose range is between about 10 g/day to about 15 g/day. Preferably, such a dose is taken before meal three times a day, most preferably once a day.

The polymer composition of the present disclosure can retain a large amount of bound potassium. The polymer binds potassium in the colon and does not release the bound potassium before the polymer is excreted in the faeces. The “large amount” herein does not indicate a capability of retaining all the bound potassium. Preferably, at least a part of the bound potassium is retained in order to achieve the therapeutic and/or preventive effects. It is desirable to retain about 5% to about 100% of the bound potassium. Preferably, the polymer composition can retain about 25% of the bound potassium. More preferably, about 50% of the bound potassium can be retained. More preferably, about 75% of the bound potassium can be retained. Most preferably, about 100% of the bound potassium can be retained. Optimally, a retaining period of the bound potassium is a time period for treatment and/or prevention with the polymer composition. In this embodiment, the polymer composition is used to bind and eliminate potassium in the gastrointestinal tract, and the retention period thereof is a retention period of the composition in the gastrointestinal tract, and preferably an average retention time in the colon.

The potassium-binding polymer is preferably not absorbed by the gastrointestinal tract. The expression “not absorbed by” and its grammatical synonyms do not mean that none of the administered polymer is not absorbed. It is desired that a certain amount of the polymer will not be absorbed. Preferably, about 90% or more of the polymer is not absorbed. More preferably, about 95% or more of the polymer is not absorbed. More preferably, about 97% or more of the polymer is not absorbed. Most preferably, about 98% or more of the polymer is not absorbed.

In some embodiments, the potassium-binding polymer contains various protic or ionic acidic groups, for example, sulfonic acid group (—SO₃—), sulfuric acid group (—OSO₃—), carboxylic group (—CO₂—), phosphonic acid group (—OPO₃²—), phosphate group (—OPO₃²—), and sulfamic acid group (—NHSO₃—). Optimally, under the physiological pH of colon, an ionization percentage of the acidic groups is greater than 75%, and the potassium-binding energy is greater than 5 mmol/g. Preferably, the ionization percentage of the acidic groups is greater than 80%, more preferably, greater than 90%, and most preferably, approximately equal to 100%.

In certain embodiments, the acid group-containing polymer contains more than one kind of acid groups. In some embodiments, the acidic group-containing polymer taken is actually an acid anhydride-based polymer, which will form an ionic polymer once it comes into contact with physiological fluids. In some other embodiments, the polymer preferably has a pKa-reducing group next to the acidic group, more preferably an electron-withdrawing substituent group, and most preferably, the pKa-reducing group is positioned at a or p position of the acidic group. Preferably, the electron-withdrawing substituent group includes hydroxyl, ether groups, ester groups, or halogen atoms, and most preferably fluorine. Preferably, the acidic group is sulfonic acid group (—SO₃—), sulfuric acid group (—OSO₃—), carboxylic group (—CO₂—), phosphonic acid group (—OPO₃²—), phosphate group (—OPO₃²—), or sulfamic acid group (—NHSO₃—). It is also preferred that the polymer can be prepared through a polymerization reaction of α-fluoroacrylic acid, difluoromaleic acid, or corresponding anhydrides thereof.

Suitable phosphonic acid monomers include vinylphosphonic acid, ethylene-1,1-bisphosphonic acid, ethylene derivative of phosphonic acid carboxylates, oligo(methylene phosphonic acid), and hydroxyethane-1,1-bisphosphonic acid. The synthesis methods of these monomers are known.

The free radical polymer prepared from monomers such as vinyl sulfonate, vinyl phosphonate, or vinyl sulfamate can be used.

The preferred monomers used herein are α-fluoroacrylate and difluoromaleic acid, and most preferably α-fluoroacrylate. These monomers can usually be prepared by a variety of methods (Gassenetal, J. Fluorine Chemistry, 55, (1991) 149-162, K F Pittman, C. U., M. Ueda, etal, Macromolecules (1980), 13(5), 1031-1036). Difluoromaleic acid is preferably prepared by oxidation of fluoroaromatic compounds (Bogachevetal, Zhurnal Organisheskoi Khimii, 1986, 22(12), 2578-83), or by oxidation of fluoro-substituted furan derivatives (U.S. Pat. No. 5,112,993). A preferred method for synthesizing α-fluoroacrylate has been disclosed in European Patent EP415214.

Other methods include step-growth polymerization of functional compounds, and the functional compound contains a phosphonic acid group, a carboxylic group, a phosphate group, a sulfinic acid group, a sulfate group, or a sulfonic acid group. For example, the high-density polyphosphonic acid sold by Rhodia (Trademark: Briquest) is very suitable for use in the present disclosure.

The polymer of the present disclosure also includes an ion exchange resin synthesized from natural polymers, and the natural polymers may be glycopolymers and semi-synthetic polymers. These polymers can be treated to have ion exchange positions on their backbone or pendant groups. For example, the glycopolymers may be materials derived from plants or animals, including cellulosic materials, hemicellulose, alkyl cellulose, hydroxyalkyl cellulose, carboxymethyl cellulose, ethyl cellulose sulfonate, starch, xylan, amylopectin, chondroitin, hyaluronate ester, heparin, guar gum, xanthan gum, mannan, galactomannan, chitin, and polyglucosamine. The most preferred polymers should not be decomposed or absorbed under the physiological conditions of the gastrointestinal tract, for example, carboxymethyl cellulose, polyglucosamine, and ethyl cellulose sulfonate.

The potassium-binding polymer can be encapsulated in a dialysis bag, paper bag, microporous matrix, polymer gel, hollow fiber, vesicle, capsule, tablet, or membrane.

The polymer can be prepared by homogeneous or heterogeneous polymerization. In the homogeneous reaction, the soluble polymer chain reacts with the cross-linking agent to obtain a cross-linked gel, and further, the cross-linked gel is extruded to form particles or pulverized into smaller particles. In the heterogeneous reaction, particles are obtained through emulsification or dispersion of soluble polymer precursors and then crosslinking. In another method, the particles are prepared by polymerizing monomers in an emulsification, suspension, microemulsification or dispersion process. The continuous phase can be an aqueous solvent or an organic solvent. When adopting the suspension process, any suitable alternative process can be used, including methods such as “template polymerization” and “multistage seeded suspension”. Almost all the particles produced by these methods are monodisperse particles. In a particular embodiment, the particles are prepared using a “spraying” process (see U.S. Pat. No. 4,427,794). In this process, a mixture of monomer and initiator is injected into a continuous phase through vibrating nozzles. The nozzles are arranged as a rapidly rotating turntable, and thus the liquid can be accelerated under centrifugal force.

Direct suspension polymerization is one preferred process for the production of α-fluoroacrylate particles. Generally, in order to prevent aggregation of particles during the production process, a suspension stabilizer is used, for example, gelatin, polyvinyl alcohol, and sodium polyacrylate, sodium hydroxymethylcellulose. It has been found that the aggregation and particle agglomeration can be reduced by adding NaCl to the aqueous phase. Other salts suitable for this purpose include various salts capable of being dissolved in the aqueous phase. In this embodiment, the added amount of the salt soluble in water ranges from about 0.1% by weight to about 10% by weight, more preferably from about 2% by weight to about 5% by weight, and most preferably from about 3% by weight to about 4% by weight.

Currently, it has been found that during the suspension polymerization of α-fluoroacrylate (such as MeFA), the characteristics of the free radical initiator have a greater impact on the quality of the suspension, including particle stability, particle yield, and retention of spherical shape, and other aspects. The use of water-insoluble free radical initiators, such as dodecyl peroxide, will result a high yield. It has been confirmed that the optimal effect will be achieved when the water solubility of the free radical initiator is less than 0.1 g/L, more preferably less than 0.01 g/L. In certain preferred embodiments, the poly-MeFA particles are generated in presence of the free radical initiator with low water solubility as well as the salt in the aqueous phase, such as NaCl.

### BRIEF DESCRIPTION OF DRAWINGS

The above and/or additional aspects and advantages of the present disclosure will become apparent and easy to understand from the description of embodiments in conjunction with the following drawing:

FIG. 1 is a diagram showing that Sodium zirconium cyclosilicate and MFA-TAIC polymer could reduce the increase of urine K⁺ induced by KCl.

FIG. 2 is a diagram showing that Sodium zirconium cyclosilicate and MFA-TAIC polymer significantly increased fecal K⁺ excretion (P<0.001, P<0.001).

### DESCRIPTION OF EMBODIMENTS

The present disclosure is described below with reference to specific embodiments. It should be noted that these embodiments are only descriptive and do not limit the present disclosure in any way.

The following abbreviations are used throughout the present disclosure:

MFA: methyl 2-fluoroacrylate; PVA: polyvinyl alcohol; BPO: benzoyl peroxide; TAIC: 15 triallyl isocyanurate; TVS: tetravinylsilane; DGDE: diethylene glycol divinyl ether; DADMS: diallyldimethylsilane; EGDMA: ethylene glycol dimethacrylate; TMPTMA: trihydroxymethylpropyl trimethacrylate; DPGDA: dipropylene glycol diacrylate; DVB: divinylbenzene; and ODE: octadiene.

A positive drug used in the present disclosure is sodium zirconium cyclosilicate 20 (Lokelma), a commercial product from AstraZenca/AndersonBrecon. Inc., batch No.: LJ2076A.

The crosslinking agents used in the present disclosure are listed in Table 1.

TABLE 1

| No. | Name | Abbreviation | Structural formula |
|---|---|---|---|
| (1) | Triallyl isocyanurate | TAIC | 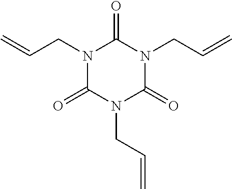 [SMILES: C=CCn1c(=O)n(CC=C)c(=O)n(CC=C)c1=O] |
| (3) | Tetravinylsilane | TVS | 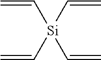 |
| (4) | 1,2,4-trivinylcyclohexane | TVCH | 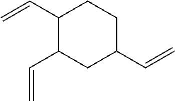 |
| (5) | Diethylene glycol divinyl ether | DGDE | 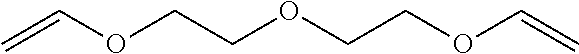 |
| (6) | Diallyldimethylsilane | DADMS | 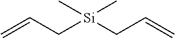 |
| (7) | Ethylene glycol dimethacrylate | EGDMA | 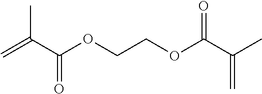 |
| (8) | Trihydroxymethylpropyl trimethacrylate | TMPTMA | 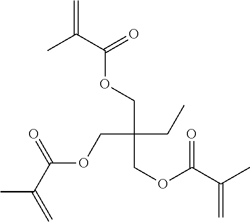 [SMILES: C=C(C)C(=O)OCC(CC)(COC(=O)C(=C)C)COC(=O)C(=C)C] |
| (9) | Dipropylene glycol diacrylate | DPGDA | 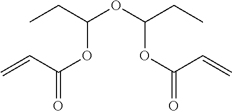 [SMILES: C=CC(=O)OC(CC)OC(CC)OC(=O)C=C] |
| (10) | Divinylbenzene | DVB | 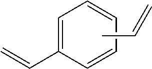 |
| (11) | Octadiene | ODE | 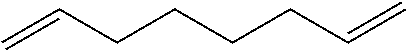 |

### Example 1

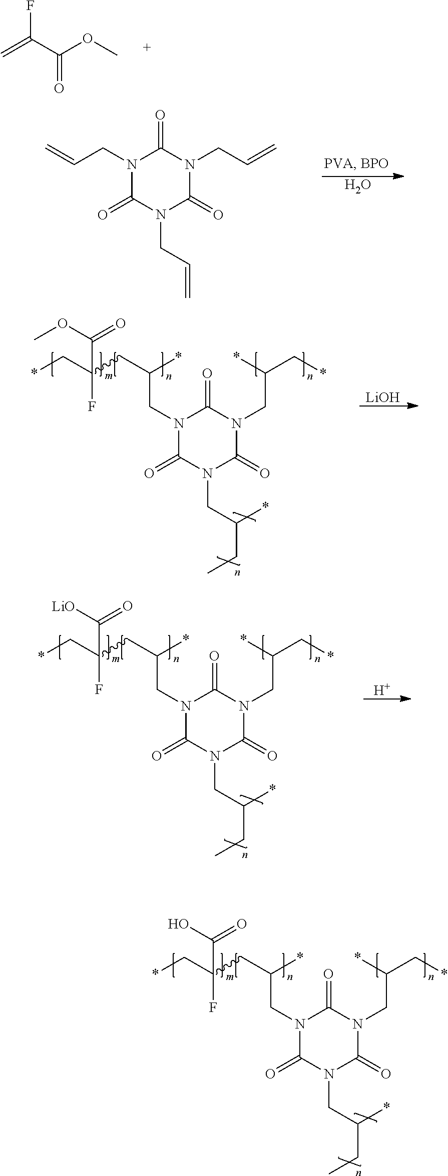

550 mL of purified water and 3.4 g of PVA were added to a reaction flask, and dissolved by stirring at 20° C. to 30° C. until they were completely dissolved clarification. MFA solution was prepared as below: 104.08 g of MWA, 14.08 g of TAIC, and 0.85 g of BPO were stirred and completely dissolved clarification for later use. The prepared MFA solution was added into the reaction flask. The reaction temperature was gradually increased to 70° C. to 80° C., followed by holding the temperature and stirring for 15 h. After the temperature was reduced to 20° C. to 30° C., suction filtration was performed. The filter cake was pulped and washed respectively with 1 L water and 1 L ethanol, 3 times each. The obtained wet product was vacuum-dried at 50° C. to obtain 101.0 g of a white solid, i.e., MFA-TAIC polymer ester.

400 mL of water, 50.4 g of lithium hydroxide monohydrate, and 150 mL of tetrahydrofuran were added to a reaction flask, and then the above MFA-TAIC polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water and ethanol. A lithium salt wet product of MFA-TAIC polymer was obtained by the filtration.

500 mL of water and 100 mL of concentrated hydrochloric acid were added to a reaction flask, the above-mentioned lithium salt wet product of MFA-TAIC polymer was added, followed by stirring for 15 h at 20° C. to 30° C. After the filtration, the filter cake was pulped and washed with water. The wet product obtained after filtration was pulped once with 500 mL of ethanol. The wet product obtained after filtration was vacuum-dried at 50° C. for 8 h to obtain 91.0 g of white dry product, which was crushed and sieved through a 120-mesh sieve, to obtain the finished product of the MFA-TAIC(m=0.95, n=0.05) polymer.

### Example 2

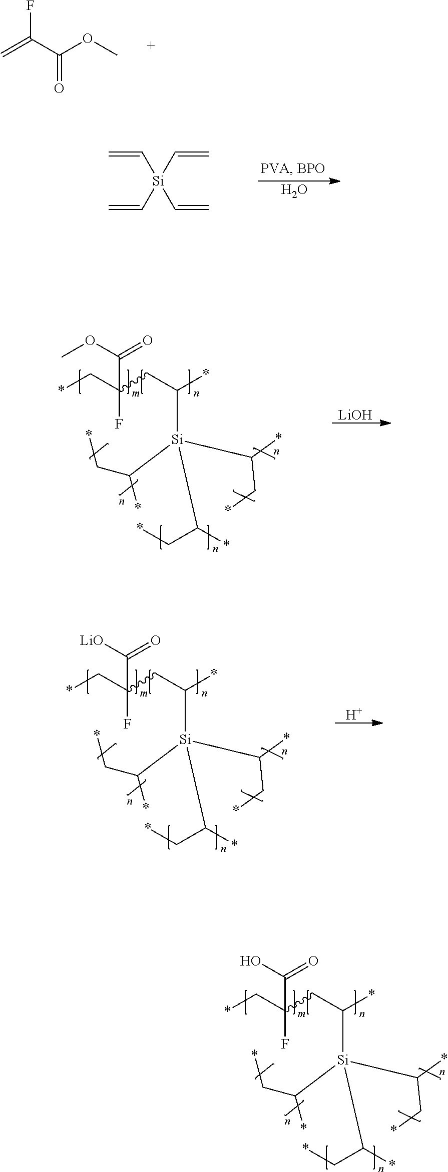

550 mL of purified water and 3.4 g of PVA were added to a reaction flask, and dissolved by stirring at 20° C. to 30° C. until they were completely dissolved clarification. An MFA solution 15 was prepared as below: 104.0 g of MFA, 6.8 g of TVS, and 0.85 g of BPO were stirred and completely dissolved clarification for later use. The prepared MFA solution was added into the reaction flask. The temperature of the materials in the reaction flask was gradually increased to 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 90.2 g of a white solid, i.e., MFA-TVS polymer ester.

400 mL of water and 50.4 g of lithium hydroxide monohydrate were added to a reaction flask, and then the above MFA-TVS polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water. The filtered wet product was a lithium salt wet product of MFA-TVS polymer.

500 mL of water and 100 mL of concentrated hydrochloric acid were added to a reaction flask, the above-mentioned lithium salt wet product of MFA-TVS polymer was added, followed by stirring for 15 h at 20° C. to 30° C. After filtration, the filter cake was repeatedly washed with water. The wet product obtained after filtration was pulped once with ethanol. The wet product obtained after filtration was vacuum-dried at 50° C. for 8 h to obtain 79.4 g of white dry product, which was crushed and sieved through a 120-mesh sieve, to obtain the finished product of the MFA-TVS (m=0.95, n=0.05) polymer.

### Example 3

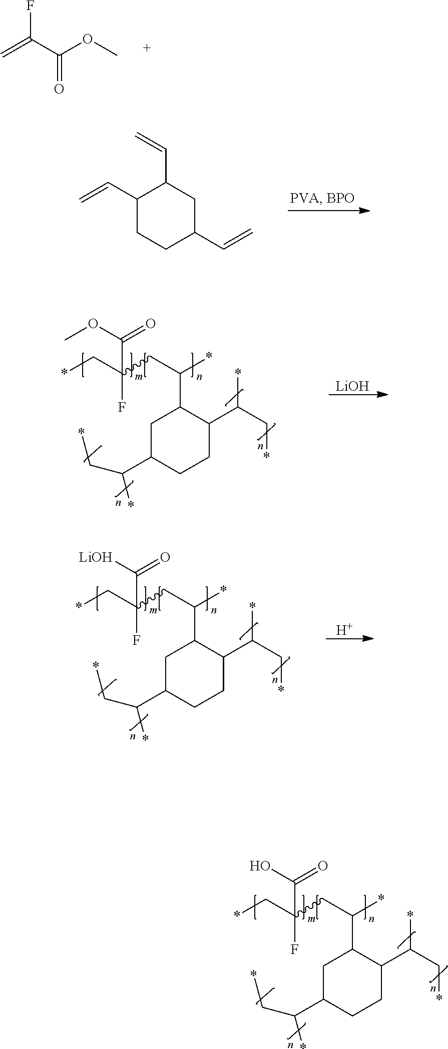

550 mL of purified water and 3.4 g of PVA were added to a reaction flask, and dissolved by stirring at 20° C. to 30° C. until they were completely dissolved clarification. An MFA solution was prepared as below: 104.0 g of MFA, 8.1 g of TVCH, and 0.85 g of BPO were stirred and completely dissolved clarification for later use. The prepared MFA solution was added into the reaction flask. The temperature of the materials in the reaction flask was gradually increased 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 101.2 g of a white solid, i.e., MFA-TVCH polymer ester.

400 mL of water and 50.4 g of lithium hydroxide monohydrate were added to a reaction flask, and then the above MFA-TVCH polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water. The filtered wet product was a lithium salt wet product of MFA-TVCH polymer.

500 mL of water and 100 mL of concentrated hydrochloric acid were added to a reaction flask, the above-mentioned lithium salt wet product of MFA-TVCH polymer was added, followed by stirring for 15 h at 20° C. to 30° C. After filtration, the filter cake was repeatedly washed with water. The wet product obtained after filtration was pulped once with ethanol. The wet product obtained after filtration was vacuum-dried at 50° C. for 8 h to obtain 82.7 g of a white dry product, which was crushed and sieved through a 120-mesh sieve, to obtain the finished product of the MFA-TVCH (m=0.95, n=0.05) polymer.

### Example 4

550 mL of purified water and 3.4 g of PVA were added to a reaction flask, and dissolved by stirring at 20° C. to 30° C. until they were completely dissolved clarification. An MFA solution was prepared as below: 104.0 g of MFA, 6.5 g of DVB, and 0.85 g of BPO were stirred and completely dissolved clarification for later use. The prepared MFA solution was added into the reaction flask. The temperature of the materials in the reaction flask was gradually increased to 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 112.0 g of a white solid, i.e., MFA-DVB polymer ester.

400 mL of water and 50.4 g of lithium hydroxide monohydrate were added to a reaction flask, and then the above MFA-DVB polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water. The filtered wet product was a lithium salt wet product of MFA-DVB polymer.

500 mL of water and 100 mL of concentrated hydrochloric acid were added to a reaction flask, the above-mentioned lithium salt wet product of MFA-DVB polymer was added, followed by stirring for 15 h at 20° C. to 30° C. After filtration, the filter cake was repeatedly washed with 4 L of water. The wet product obtained after filtration was pulped once with 500 mL of ethanol. The wet product obtained after filtration was dried at 50° C. for 8 h to obtain 82.3 g of a white dry product, which was crushed and sieved through a 120-mesh sieve, to obtain the finished product of the MFA-DVB (m=0.95, n=0.05) polymer.

### Example 5

550 mL of purified water and 3.4 g of PVA were added to a reaction flask, and dissolved by stirring at 20° C. to 30° C. until they were completely dissolved clarification. An MFA solution was prepared as below: 104.0 g of MFA, 5.5 g of ODE, and 0.85 g of BPO were stirred and completely dissolved clarification for later use. The prepared MFA solution was added into the reaction flask. The temperature of the materials in the reaction flask was gradually increased to 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 107.0 g of a white solid, i.e., MFA-ODE polymer ester.

400 mL of water and 50.4 g of lithium hydroxide monohydrate were added to a reaction flask, and then the above MFA-ODE polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water. The filtered wet product was a lithium salt wet product of MFA-ODE polymer.

500 mL of water and 100 mL of concentrated hydrochloric acid were added to a reaction flask, the above-mentioned lithium salt wet product of MFA-ODE polymer was added, followed by stirring for 15 h at 20° C. to 30° C. After filtration, the filter cake was repeatedly washed with 4 L of water. The wet product obtained after filtration was pulped once with 500 mL of ethanol. The wet product obtained after filtration was dried at 50° C. for 8 h to obtain 80.3 g of a white dry product, which was crushed and sieved through a 120-mesh sieve, to obtain the finished product of the MFA-ODE (m=0.95, n=0.05) polymer.

### Example 6

550 mL of purified water and 3.4 g of PVA were added to a reaction flask, and dissolved by stirring at 20° C. to 30° C. until they were completely dissolved clarification. An MFA solution was prepared as below: 104.0 g of MFA, 14.0 g of TAIC, and 0.85 g of BPO were stirred and 20 completely dissolved clarification for later use. The prepared MFA solution was added into the reaction flask. The temperature of the materials in the reaction flask was gradually increased to 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 101.0 g of a white solid, i.e., MFA-TAIC polymer ester.

400 mL of water and 48.0 g of sodium hydroxide were added to a reaction flask, and then the above MFA-TAIC polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with 1 L of water 3 times. The filtered wet product was a sodium salt wet product of MFA-TAIC polymer. The wet product obtained after filtration was dried at 50° C. for 12 h to obtain 93.7 g of a white dry product, which was crushed and sieved through a 120-mesh sieve, to obtain the finished product of the MFA-TAIC (m=0.95, n=0.05) polymer.

### Example 7

550 mL of purified water and 3.4 g of PVA were added to a reaction flask, and dissolved by stirring at 20° C. to 30° C. until they were completely dissolved clarification. An MFA solution was prepared as below: 104.0 g of MFA, 14.0 g of TAIC, and 0.85 g of BPO were stirred and completely dissolved clarification for later use. The prepared MFA solution was added into the reaction flask. The temperature of the materials in the reaction flask was gradually increased to 70° C. to 80° C., followed by holding the temperature and stirring for 15 h. After the temperature was reduced to 20° C. to 30° C., suction filtration was performed. The filter cake was pulped and washed with water. The obtained wet product was dried to obtain 101.0 g of a white solid, i.e., MFA-TAIC polymer ester.

400 mL of water and 48.0 g of sodium hydroxide were added to a reaction flask, and then the above MFA-TAIC polymer ester was added under stirring. The temperature was increased to 50° C. to 60° C., followed by stirring and holding the temperature for 15 h. The temperature was lowered to 20° C. to 30° C., then filtration was performed, and the filter cake was pulped and washed with water. The filtered wet product was a sodium salt wet product of MFA-TAIC polymer.

The filtered wet product was added to 1 L of water, 333.0 g of anhydrous calcium chloride was added, followed by pulping and stirring at 20° C. to 30° C. for 6 h. The filtered wet product was repeatedly pulped and washed with 1 L water 3 times. The filtered wet product was added with 1 L of water and 333.0 g of anhydrous calcium chloride, followed by pulping and stirring at 20° C. to 30° C. for 6 h. The filtered wet product was repeatedly pulped with 1 L of water until the conductivity was ≤50 μs/cm. After suction filtration, the wet product was dried at 50° C. for 18 h to obtain 99.2 g of a white dry product, which was crushed and sieved through a 120-mesh sieve, to obtain the finished product of the MFA-TAIC calcium salt (m=0.95, n=0.05) polymer.

### Example 8

Potassium buffer: potassium buffer was prepared with 150 mmol/L potassium and 200 mmol/L 2-[morpholino] ethanesulfonic acid, the pH was 6.0 to 8.0.

Standard graph: Identify five 100 m1 volumetric flasks by the numbers 1, 2, 3, 4, and 5. In that order pipet 1, 3, 6. 8, and 10 mL of potassium buffer into the flasks, dilute with water to volume, and mix. Perform ion chromatography detection on volumetric flasks 1, 2, 3, 4, and 5 and record the peak area of potassium ion. On ruled coordinate paper, plot the observed peak area as the ordinate, and the concentrations, in mmol per liter, of potassium as the abscissa.

Test sample solution: Take about 1.6 g of polymer, place it in a 250 m1 Erlenmeyer flask, add 100 m1 of potassium buffer, water bath at 37° C. 2° C., stir with magnet for 24 h, shake evenly, sample (15 min, 3 h, 5 h or 24 h as recommended), filter, precisely pipet 1 ml of filtrate into a 100 m1 volumetric flask, and dilute to the mark with water.

Analyze test sample solution by ion chromatography and record the peak area of potassium ion, and determine the potassium concentration, in mmol per liter, by interpolation from the Standard graph. Calculate the adsorption amount, in mmol per g, of potassium ion adsorbed on the resin taken by the formula:

Potassium ion adsorption amount of polymer=(X−2.5Y)/W

in which X is the weight, in mmol, of potassium in 100 mL of Potassium solution before exchange; Y is the weight, in mmol, of potassium per L as interpolated from the Standard graph; and W is the weight, in g, of polymer taken, expressed on the anhydrous basis.

The chromatographic conditions are listed in Table 2 below.

TABLE 2

| | |
|---|---|
| Chromatographic | CS16, CS17, CS12A (250 mm × 4 mm) |
| column | |
| protection column | CG16, CG17, CG12A (50 mm × 4 mm) |
| Detector | Electrical conductivity detector |
| Suppressor | CSRS 4 mm |
| Flow rate | 0.3 to 5 ml/min, preferably 1.0 ml/min |
| Column temperature | 30° C. |
| Current | 18 mA |
| Detector temperature | 35° C. |
| Injection volume | 10 to 100 μl, preferably 10 μl |
| Eluent | Methanesulfonic acid solution, preferably 6 mM |
| Running time | 20 min |

Results and Analysis

The potassium ion adsorption capacities of the polymers in Examples 1, 2, 3 and 5 were measured with this method, as shown in Table 3 below.

TABLE 3

| | Potassium ion adsorption |
|---|---|
| | amount of polymer |
| Example No. | (mmol/g) |
| Example 1 (MFA-TAIC) | 7.5 |
| Example 2 (MFA-TVS) | 7.5 |
| Example 3 (MFA-TVCH) | 7.7 |
| Example 5 (MFA-ODE) | 6.7 |
| Sodium zirconium cyclosilicate | 3.6 |
| (commercial product from | |
| AstraZenca) | |
| Veltassa (commercial | 2.6 |
| product from Relypsa) | |

Conclusion: It can be seen from the above table that the polymer prepared by using the crosslinking agent with three or four reaction sites in the present disclosure has a significantly higher potassium ion-adsorption capacity than the polymer prepared by using the crosslinking agent with two reaction sites, and significantly higher than the polymers known in the related art.

### Example 9

The samples listed in Table 4 below were placed in an aluminum plastic bag for Stability test (25° C.±2° C./60% RH±5% RH, 40° C.±2° C./75% RH±5% RH), and the results are shown in Table 4.

TABLE 4

| | | | Accelerated | Accelerated |
|---|---|---|---|---|
| | | Free fluoride | stability test | stability test |
| Material | | ion content | (25° C. ± 2° C./ | (40° C. ± 2° C./ |
| name | Process | (μg/g) (day 0) | 60% RH ± 5% RH) | 75% RH ± 5% RH) |
| Example 1 | MFA-TAIC | 36.0 | Placed at 25° C. for 2 | Placed at 40° C. for 2 |
| | | | months, the free | months, the free |
| | | | fluoride ion content | fluoride ion content |
| | | | not increased | not increased |
| Veltassa | Commercial | 14.3 | Placed at 25° C. for 2 | Placed at 40° C. for 2 |
| | product from | | months, the free | months, the free |
| | Relypsa (batch | | fluoride ion content | fluoride ion content |
| | number | | significantly | significantly |
| | CCWVX) | | increased, 8.0 times | increased, 25.1 times |
| | | | that on day 0 | that on day 0 |

It can be seen from the above table that the polymers of the present disclosure have high stability, which is significantly higher than the polymer of the related art.

### Example 10

32 normal male SD rats were reared adaptively for 3-5 days and then randomly divided into 4 groups, i.e., a normal group, a model group, a positive control group (sodium zirconium cyclosilicate), a MFA-TAIC polymer group, each group including 8 rats. In accordance with intragastric administration at a volume of 20 m1/kg, the normal group was administered with a corresponding solvent (0.1% xanthan gum), the positive control group was administered with sodium zirconium cyclosilicate at 1.5 g/kg, the MFA-TAIC polymer group was administered with 10 MFA-TAIC polymer at 1.5 g/kg, for only one time. The model group, the positive control group (sodium zirconium cyclosilicate), and the MFA-TAIC polymer group were respectively intraperitoneally injected with 10% KCl at a volume of 4 m1/kg 3 hours after the administration, and then supplemented with 5% KCl solution every 1 hour, continuously for 8 hours, i.e., supplemented with KCl solution 8 times. Before the administration, 3 hours after the administration (i.e., before the modeling (0 h)), and 1 h, 2 h, 4 h, 6 h, and 8 h after the modeling, 0.4 mL of whole blood was collected from the orbit, serum was separated, and blood potassium level was detected. All animals were individually placed in metabolic cages, and urine and faeces within 24 hours after administration were collected on the next day to detect potassium ion content. If the blood collection and KCl injection had conflicts in term of time, the blood was collected 2 minutes in advance, and then KCl was intraperitoneally injected on time after the blood collection. If deaths occurred, the time and number of deaths were recorded. The faeces were freeze-dried, and the weight of dried faeces of each rat was recorded. Cations in the faeces were extracted with 1M HCl overnight. Then, the ion chromatography analysis was performed.

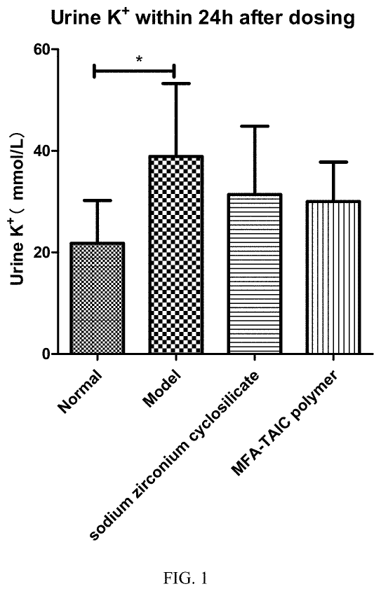

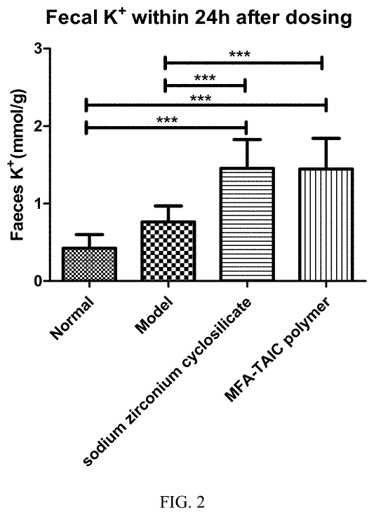

The results showed that compared with the normal group, the urine K⁺ level in the model group was significantly increased (P<0.05), and sodium zirconium cyclosilicate and MFA-TAIC polymer could reduce the increase of urine K⁺ induced by KCl, as shown in FIG. 1. Compared with normal group, K⁺ in faeces of model group increased to a certain extent, increasing by 79.87%, with no statistical difference. Compared with the normal group, sodium zirconium cyclosilicate and MFA-TAIC polymer increased fecal K⁺ excretion (P<0.001, P<0.001), and compared with the model group, sodium zirconium cyclosilicate and MFA-TAIC polymer increased fecal K⁺ excretion (P<0.001, P<0.001), as shown in FIG. 2.

In the specification, descriptions with reference to the terms “an embodiment”, “some embodiments”, “examples”, “specific examples”, or “some examples”, etc. mean that specific features, structures, materials or characteristics described in conjunction with the embodiment or example are included in at least one embodiment or example of the present disclosure. In this specification, the above terms are illustrative, and do not necessarily refer to the same embodiment or example. Moreover, the described specific features, structures, materials or characteristics can be combined in a suitable manner in any one or more embodiments or examples. In addition, those skilled in the art can combine different embodiments or examples and the features of the different embodiments or examples described in this specification without contradicting each other.

Although the embodiments of the present disclosure are illustrated and described above, it can be understood that the above-mentioned embodiments are illustrative and should not be construed as limitations of the present disclosure. Those skilled in the art can make changes, modifications, substitutions, and variations to the above-mentioned embodiments within the scope of the present disclosure.

1. A polymer, comprising repeating units obtained by polymerizing a monomer and a crosslinking agent, wherein a molar ratio of the monomer to the crosslinking agent ranges from 1:0.02 to 1:0.20, and the monomer comprises an acidic group and a pKa-reducing group next to the acidic group, wherein the acidic group is selected from the group consisting of a sulfonic acid group (—SO₃—), a sulfuric acid group (—OSO₃—), a carboxylic group (—CO₂—), a phosphonic acid group (—OPO₃²—), a phosphate group (—OPO₃²—), and a sulfamic acid group (—NHSO₃—); the pKa-reducing group is selected from the group consisting of nitro, cyano, carbonyl, trifluoromethyl, and halogen atoms; and the crosslinking agent has three or four reaction sites.

2. The polymer according to claim 1, wherein the acidic group is the carboxylic group, and the pKa-reducing group is fluorine.

3. The polymer according to claim 1, wherein the reaction sites are free alkenyl groups.

4. The polymer according to claim 1, wherein the crosslinking agent comprises at least one of triallyl isocyanurate, 1,2,4-trivinylcyclohexane, tetravinylsilane, trimethylolpropane trimethacrylate, triallyl cyanurate, or triallylcarbinol.

5. The polymer according to claim 1, wherein the polymer is at least one selected from the group consisting of polyvinyl sulfonic acid polymer, polyvinyl sulfamic acid polymer, poly(vinyl sulfamic acid/vinyl sulfuric acid) copolymer, polyvinyl amino phosphonic acid polymer, N-(bisphosphonate ethyl) polyvinylamine polymer, poly(α-fluoroacrylic acid) polymer, vinylphosphonic acid/acrylic acid copolymer, vinylphosphonic acid/α-fluoroacrylic acid copolymer, polyvinylsulfuric acid polymer, and cross-linked polyvinylsulfamic acid polymer.

6. A polymer, having a structure represented by formula (I) or being a salt of the structure represented by formula (I): 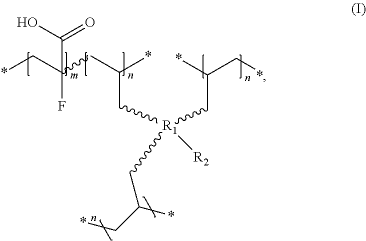
wherein, R₁ is 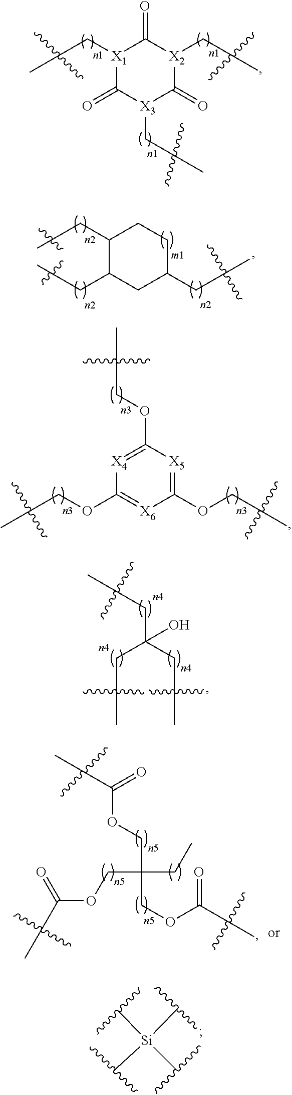
when R₁ contains three binding sites, R₂ is not present; and when R₁ contains four binding sites, R₂ is connected to one of the four binding sites of R₁ and R₂ is 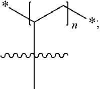
m ranges from 0.80 to 0.98, n ranges from 0.02 to 0.20, and m+n=1;
n₁, n₂, n₃, n₄, and n₅ are each independently selected from 0, 1, 2, 3, 4, 5, 6, or 7;
X₁, X₂, X₅, X₄, X₅, and X₆ are each independently a carbon atom or a nitrogen atom;
m1 is 0 or 1; and
* represents a binding site.

7. The polymer according to claim 6, wherein R₁ is

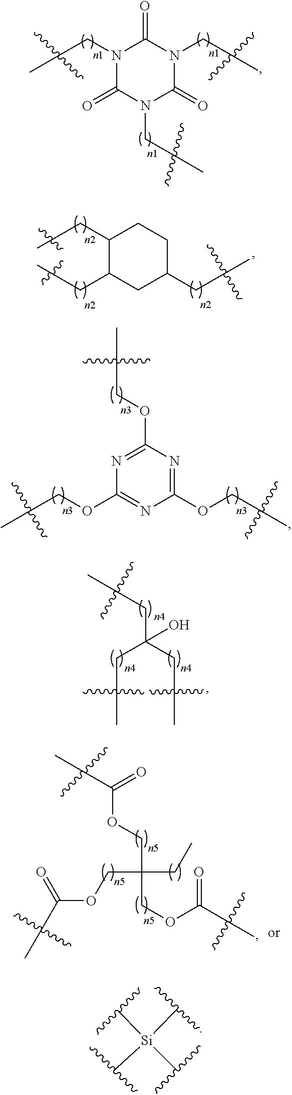

8. The polymer according to claim 6, wherein R₁ is

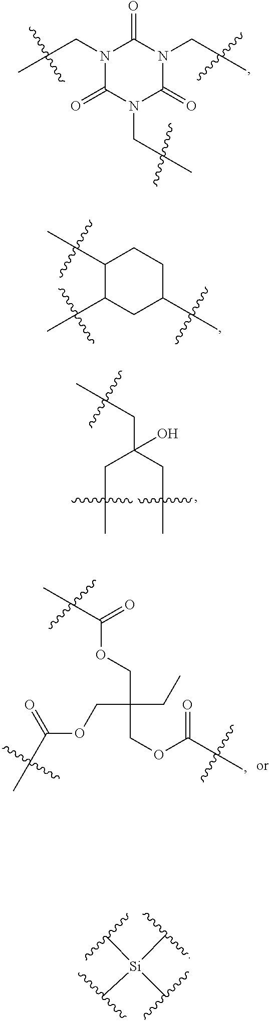

9. A polymer, having any one of the following structures or being a salt of any one of the following structures: 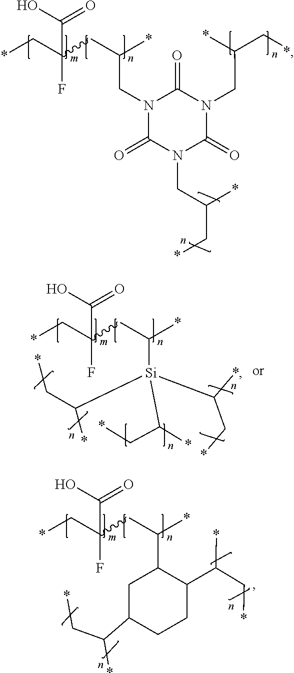
wherein m ranges from 0.80 to 0.98; n ranges from 0.02 to 0.20; and m+n=1.

10. A pharmaceutical composition, comprising the polymer according to claim 1, and a pharmaceutically acceptable excipient.

11. A method for reducing potassium in a subject, comprising administrating the polymer according to claim 1 to the subject.

12. A method for treating or preventing hyperkalemia in a subject, comprising administrating a therapeutic effective amount of the polymer according to claim 1 to the subject.

13. The method according to claim 12, wherein the hyperkalemia is caused by administration of a medicament that causes potassium retention.

14. The method according to claim 13, wherein the medicament that causes potassium retention is selected from the group consisting of non-potassium diuretics, angiotensin-converting enzyme inhibitors, non-steroidal anti-inflammatory drugs, heparin, and trimethoprim.

15. The method according to claim 13, wherein the polymer is in a form of a pharmaceutical composition containing a pharmaceutically acceptable excipient.

16. A potassium ion adsorption determination method, comprising: detecting an adsorption capacity of the polymer according to any claim 1 for potassium ions through ion chromatography.

17. A method for treating or preventing hyperkalemia in a subject, comprising administrating a therapeutic effective amount of the polymer according to claim 6 to the subject.

18. The method according to claim 17, wherein the hyperkalemia is caused by administration of a medicament that causes potassium retention.

19. A method for treating or preventing hyperkalemia in a subject, comprising administrating a therapeutic effective amount of the polymer according to claim 9 to the subject.

20. The method according to claim 19, wherein the hyperkalemia is caused by administration of a medicament that causes potassium retention.

21. A pharmaceutical composition, comprising the polymer according to claim 6, and a pharmaceutically acceptable excipient.

22. A pharmaceutical composition, comprising the polymer according to claim 9, and a pharmaceutically acceptable excipient.
